(19) United States
(12) Patent Application Publication
(10) Pub. No.: US 2006/0189817 A1
(43) Pub. Date: Aug. 24, 2006

(54) Method for producing conjugated linoleic acid

(76) Inventors: Peter Horlacher, Bellenberg (DE); Karl-Heinz Ruf, Babenhausen (DE); Franz Timmermann, Illerlissen (DE); Wolfgang Adams, Meckenbeuren (DE); Rainer V. Kries, Illertissen (DE)

Correspondence Address: COGNIS CORPORATION;PATENT DEPARTMENT, 300 BROOKSIDE AVENUE, AMBLER, PA, 19002, US

(21) Appl. No.: 10/538,383

(22) PCT Filed: Dec. 9, 2003
(86) PCT No.: PCT/EP03/13922

§ 371 Date: Jun. 13, 2005

(30) Foreign Application Priority Data
Dec. 18, 2002 (DE) 102 59 157.1

(51) Int. Cl. C07C 51/347

(52) U.S. Cl. 554/126

(57) ABSTRACT

The present invention relates to a process for the production of conjugated linoleic acid, in which (a) linoleic acid lower alkyl esters containing 1 to 5 carbon atoms in a linear or branched alkyl chain are isomerized in the presence of alkali metal alcoholates, (b) the then conjugated linoleic acids are released from the ester after saponification, and (c) the free linoleic acids are subjected to crystallization. The crystallization step of said method provides a cost-effective option for producing conjugated linoleic acids with a high purity and excellent storage stability, in particular in low temperatures.

### FIELD OF THE INVENTION

This invention relates generally to fatty acids and, more particularly, to a new process for the production of enriched conjugated linoleic acid which does not lead to product clouding at temperatures below 10° C.

### PRIOR ART

Several methods for the production of polyunsaturated linoleic acids containing conjugated double bonds, which are marketed under the name of “CLA” (conjugated linoleic acid) and which are being increasingly used as food additives, are described in the literature.

Pure CLA is generally obtained by saponification of oils containing linoleic acid. The disadvantage of these processes lies in the high percentage of unwanted isomers and the poor yield.

Thus, International patent application WO 96/06605 A1 describes the production of free conjugated linoleic acid from triglycerides and linoleic acid which are saponified with potassium hydroxide in ethylene glycol and, at the same time, isomerized. After neutralization of the reaction mixture with hydrochloric acid, the CLA is extracted with an organic solvent (hexane) and purified by washing.

European patent EP 0 839 897 B1 discloses a process in which triglycerides containing linoleic acid are subjected to an alkaline isomerization with potassium hydroxide in propylene glycol. Further working up is again carried out by neutralization with hydrochloric acid, extraction with hexane and washing with 5% by weight NaCl solution.

A similar process for the production of a high-quality CLA is described in European patent application EP 0 950 410 A1.

In the production of the conjugated linoleic acid mentioned above, safflower oil (thistle oil) or sunflower oil is used as the starting material. A major disadvantage of this process is that, on the one hand, the content of conjugated linoleic acid is determined by the linoleic acid content of the trigly\grave{c}erides (normally contents of only 63-78% can be obtained) and, on the other hand, a large quantity of unwanted waste materials is formed. Another disadvantage of these products lies in the high content of saturated fatty acids (above all palmitic and stearic acid), so that the product contains deposits at relatively low temperatures (<10° C.).

In order to overcome these disadvantages, the content of saturated fatty acids is generally reduced by repeated distillation. However, this leads to distinct losses of yield which is undesirable for economic and ecological reasons.

Accordingly, the problem addressed by the present invention was to provide a process for the production of conjugated linoleic acid which would be distinguished by high profitability and which would give high yields of a high-purity end product. The conjugated linoleic acid produced would be distinguished by good stability in storage and would not have any tendency to precipitate, particularly at low temperatures.

### DESCRIPTION OF THE INVENTION

The present invention relates to a process for the production of conjugated linoleic acid, in which
(a) linoleic acid lower alkyl esters containing 1 to 5 carbon atoms in a linear or branched alkyl chain are isomerized in the presence of alkali metal alcoholates,
(b) the then conjugated linoleic acids are released from the ester after saponification and
(c) the free linoleic acids are subjected to crystallization.

It has surprisingly been found that conjugated linoleic acid with improved purity and improved storage stability at low temperatures can be obtained in high yields by this process.

The production from linoleic acid lower alkyl esters affords the possibility of obtaining products with a CLA content of >78%. The insertion of a crystallization step results in a far more economical process than conventional process steps, such as distillation, where losses of yield of the order of >20% occur.

The content of unwanted secondary products, such as palmitic acid, can be reduced by fractional distillation of the linoleic acid lower alkyl esters to such an extent that the content of conjugated linoleic acid amounts to more than 78% by weight. Accordingly, the disadvantage of this process lies on the one hand in the fractional distillation yield and, on the other hand, in unwanted secondary products (lower alcohol, residual ester contents). These disadvantages were obviated by using a crystallization step instead of distillation. A storage-stable end product was obtained and could be stored at low temperatures—temperatures below 10° C.—without any precipitations.

Conjugated Linoleic Acid (CLA)

In the context of the invention, conjugated linoleic acid is preferably understood to mean the main isomers 9 cis, 11 trans octadecadienoic acid and 10 trans, 12 cis, but also the isomer mixtures which are normally formed in the production of conjugated linoleic acid. The raw materials produced by the process according to the invention are themselves intended to contain a high percentage of the preferred isomers.

Linoleic Acid Lower Alkyl Esters

Suitable starting materials for the process according to the invention are linoleic acid lower alkyl esters which preferably correspond to formula

(I):
R¹CO—OR² (I)
in which R¹CO is the acyl residue of a linoleic acid and R² is a linear or branched C₁₋₅ alkyl group. Conjugated linoleic acid methyl and/or ethyl esters in particular are used.
Isomerization

The isomerization of the linoleic acid lower alkyl esters is carried out with alkali metal alcoholates in an inert gas atmosphere at temperatures of 90 to 150° C., preferably at temperatures of 100 to 130° C. and more particularly at temperatures of 105 to 125° C.

In a preferred embodiment, C₁₋₁₀ alkali metal alcoholates are used as bases during the isomerization, potassium methanolate, potassium ethanolate and potassium t-butylate being particularly preferred.

Saponification

The saponification of the isomerized linoleic acid lower alkyl esters with aqueous alkali metal hydroxide solutions is carried out at temperatures of 40 to 90° C., preferably at temperatures of 60 to 80° C. and more particularly at temperatures of 65 to 75° C. It is continued to a degree of hydrolysis of 80 to 100% by weight and preferably above 98%.

Release of the CLA

After the treatment with the alkali metal hydroxides, the CLA is present as a soap and is released by acidification with phosphoric acid or citric acid. This is followed by phase separation, in which the aqueous phase is removed, and the supernatant solution is isolated. This supernatant solution contains the CLA and is again washed with water and then dried in vacuo.

Crystallization

The most important process step in terms of process economy is crystallization. Both the palmitic acid content and the stearic acid content are reduced by crystallization. The stearic acid is a determining factor in the formation of deposits at relatively low temperatures. The crystallization step may be carried out one or more times depending on the quality of the conjugated linoleic acid. It is carried out at temperatures below 10° C. and preferably at temperatures below 6° C.

### EXAMPLE

### Production of Conjugated Linoleic Acid

1,190 g linoleic acid ethyl ester from safflower oil were introduced into a heatable flask and 60 g potassium ethanolate (32% by weight) were added with stirring under nitrogen at 110° C., ethanol being continuously distilled off. After addition of 190 g water, 1,070 g of a 25% by weight potassium hydroxide solution were pumped into the flask for saponification.

To release the free acid, 770 g water were added to the mixture and 510 g phosphoric acid (85% by weight) were then introduced at 70° C. For phase separation, the mixture was then left standing for 30 minutes at 70° C. After the aqueous phase had been removed, the mixture was again washed with water and, finally, the organic phase was dried in vacuo.

After saponification, the free conjugated linoleic acid were crystallized.

Crystallization (Step C)

System Specification:

Control: PC with Wizcon program
Heating/cooling: Julabo FP 50 thermostat
Crystallization vessel: 17.8 liter capacity DeSmet crystallizer (DeSmet, Belgium)
Stirrer: wall-sweeping anchor stirrer, total vessel height
Filter press: coolable filter press, temperature as bath temp., chamber: diameter 20 cm, width 1.8 cm, capacity 565 cm³
   Procedure:

The fatty acid was introduced into the crystallizer and cooled to 5° C.

TABLE 1
Crystallization sequence

| | | Product | |
|---|---|---|---|
| Time | Bath temp. | temp. | Remarks |
| 0:42 | 3.0° C. | 5.3° C. | Very good crystals, beginning |
| | | | of filtration |
| 0:44 | 3.0° C. | 5.3° C. | Filtrate begins to run off |
| 0:59 | 3.0° C. | 5.4° C. | 2 liters filtrate, 0.2 bar in vessel |
| | | | 1, remove sample |
| 1:16 | 3.0° C. | 5.5° C. | 4 liters filtrate, 0.4 bar in vessel |
| | | | 2, remove sample |
| 1:25 | 3.0° C. | 5.6° C. | End of filtration, purge briefly |
| | | | with N₂, then press |

The yield after crystallization was 91%. After removal of the oligomers by distillation, the yield amounted to 86% by weight. Even with concentration by distillation, overhead distillation was again necessary at the end in order to remove oligomeric CLAs, so that a further loss of yield occurred.

Analysis:

GC method: silylation of the free fatty acid with subsequent column separation

TABLE 2
Composition of the reaction products
before and after crystallization

| | Starting | After |
|---|---|---|
| FA Spectrum | material | crystallization |
| Fatty acid C12 | <0.1 | <0.1 |
| Fatty acid C14 | 0.1 | 0.1 |
| Fatty acid C15 | <0.1 | <0.1 |
| Fatty acid C16 | 6.6 | 4.4 |
| Fatty acid C16* | 0.1 | 0.1 |
| Fatty acid C17 | <0.1 | <0.1 |
| Fatty acid C18 | 2.5 | 1.3 |
| Fatty acid C18* | 13.5 | 14.1 |
| Fatty acid C18** | 0.9 | 0.9 |
| Fatty acid C20 | 0.3 | 0.2 |
| Fatty acid C20* | 0.2 | 0.2 |
| Fatty acid C22 | 0.2 | 0.1 |
| Fatty acid C22* | <0.1 | <0.1 |
| Fatty acid C24 | 0.1 | 0.1 |
| Fatty acid C24* | 0.1 | 0.1 |
| Fatty acid C18 conjug. | 72.6 | 75.7 |
| Fatty acid C18 conjug. isomer 1 | 34.5 | 36.2 |
| Fatty acid C18 conjug. isomer 2 | 35.6 | 37.2 |
| Fatty acid C18 conjug. isomer 3 | 1.5 | 1.6 |
| Fatty acid C18 conjug. isomer 4 | 0.8 | 0.7 |
| Total fatty acid free | 97.3 | 97.4 |

The starting material (normal end product) corresponds to the production of the conjugated linoleic acid from safflower oil. It was compared with the end product after crystallization. The Example shows that the total CLA content can be enriched by the crystallization step.

If the Example in column 2 were distilled, the yield—for comparable quality (CLA content)—would be ca. 79% compared to 86% with crystallization.

Clouding Behavior at 8° C.

Both products (starting material without crystallization and crystallized material) were stored at 8° C. Only the crystallized product remained free from clouding, even after storage for several weeks.

1-3. (canceled)

4. A high yield process for the production of conjugated linoleic acid having a high degree of conjugated double bonds, purity and cold temperature stability, said process comprising
(a) isomerizing linoleic acid lower alkyl esters containing 1 to 5 carbon atoms in a linear or branched alkyl chain in the presence of an alkali metal alcoholate to yield highly conjugated linoleic acid esters,
(b) saponifying the highly conjugated linoleic acid esters to yield the highly conjugated linoleic acids, and
(c) crystallizing the highly conjugated linoleic acids one or more times.

5. A process according to claim 4, wherein the crystallization step (c) is carried out at temperatures below 10° C.

6. A process according to claim 4, wherein that the crystallization step (c) is carried out at temperatures below 6° C.

7. A process according to claim 4, further comprising distilling the crystallized highly conjugated linoleic acids obtained in step (c) to remove oligomer impurities.

8. A process according to claim 4, wherein the isomerization step (a) is carried out at temperatures of 100 to 130° C.

9. A process according to claim 4, wherein the linoleic acid lower alkyl esters have the formula (I):
R¹CO—OR² (I)
in which R¹CO is the acyl residue of a linoleic acid and R² is a linear or branched C₁₋₅ alkyl group.

10. A process according to claim 9, wherein acyl residues of a linoleic acid are conjugated.
